SEQUENCE LISTING (4 sequences)

SEQ ID NO: 1 - RNA, 19 nt, AEQUORIA VICTORIA
cuacaacagccacaacguc

SEQ ID NO: 2 - RNA, 19 nt, AEQUORIA VICTORIA
caacaucucgacaccagca

SEQ ID NO: 3 - RNA, 19 nt, AEQUORIA VICTORIA
cagccacaacgucuauauc

SEQ ID NO: 4 - RNA, 19 nt, AEQUORIA VICTORIA
acagaccaccgugucuaac

(19) United States
(12) Patent Application Publication
(10) Pub. No.: US 2005/0026286 A1
(43) Pub. Date: Feb. 3, 2005

(54) Methods and compositions for selective RNAi mediated inhibition of gene expression in mammal cells

(76) Inventors: Jen-Tsan Chi, Mountain View, CA (US); Howard Y. Chang, Burlingame, CA (US); Patrick Brown, Stanford, CA (US)

Correspondence Address: BOZICEVIC, FIELD & FRANCIS LLP, 1900 UNIVERSITY AVE, SUITE 200, EAST PALO ALTO, CA, 94303, US

(21) Appl. No.: 10/794,767

(22) Filed: Mar. 4, 2004

Related U.S. Application Data
(60) Provisional application No. 60/452,379, filed on Mar. 5, 2003.

(51) Int. Cl. A61K 48/00; C12Q 1/68; C12N 15/85

(52) U.S. Cl. 435/455; 514/44

(57) ABSTRACT

RNAi arrays and methods for using the same are provided. The subject arrays are characterized by having two or more distinct RNAi agents. The arrays find use in methods where cells are contacted with the arrays and the activity of the RNAi agents is determined by evaluating the contacted cells. The subject arrays and methods find use in a variety of applications, such as high throughput loss of function genomic applications.

### CROSS-REFERENCE TO RELATED APPLICATIONS

This application claims priority (pursuant to 35 U.S.C. § 119 (e)) to the filing date of the U.S. Provisional Patent Application Ser. No. 60/452,379 filed Mar. 5, 2003; the disclosure of which is herein incorporated by reference.

### INTRODUCTION

1. Field of the Invention

The field of this invention is RNA interference.

2. Background of the Invention

Double-stranded RNA induces potent and specific gene silencing through a process referred to as RNA interference (RNAi) or posttranscriptional gene silencing (PTGS). RNAi is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger. For a review of the RNAi process, see Paddison & Hannon, Cancer Cell (2002) 2:17-23.

RNAi has become the method of choice for loss-of-function investigations in numerous systems, including C. elegans, Drosophila, fungi, plants, and even mammalian cell lines. In such assays, RNAi agents corresponding to the gene of interest, e.g., synthetic double stranded siRNA molecules having a sequence homologous to a sequence found in a target mRNA transcribed from the gene of interest, are introduced into a cell that contains the gene of interest and the phenotype of the cell is then determined. Any deviation in observed phenotype to the control wild type phenotype is then used as a determination of function of the gene of interest, since the observed phenotype results from the siRNA mediated inactivation of the gene of interest.

In addition to the above applications, RNAi has potential in therapeutic applications. However, one of the cardinal features of RNAi in C. elegans are the potency and persistence of gene silencing. RNAi can be successfully induced in C. elegans with a few molecules of the trigger dsRNA per cell, and the silencing effect is propagated to the progeny of the treated animals. These results suggested the presence of amplification mechanisms in RNAi. Recently, “degradative PCR” was proposed as a mechanism underlying amplification in RNAi in Drosophila embryos and C. elegans. In this model, the antisense strand of siRNA hybridizes to the target mRNA and primes a RNA-dependent RNA polymerase (RdRP) reaction to generate double stranded RNA 5′ of sense sequence. The newly synthesized dsRNA molecules are then subject to Dicer digestion and generate many secondary siRNAs from the extended regions that can target additional mRNA molecules for degradation. This model explains the catalytic efficiency and potency of RNAi, but it presents potential problems for the specificity of RNAi as a research, and therapeutic, tool. A consequence of the generation of the secondary siRNAs is the spreading of the RNAi specificity to sequences 5′ to the original target sequence in the mRNA. This phenomenon, termed transitive RNAi, has been demonstrated in vivo in C. elegans. Transitive RNAi poses the possibility of silencing of a significant number of unintended genes within the genome with each siRNA experiments, and it is a greater concern with human cells due to the increased complexity of domain structures in the human proteome.

Relevant Literature

Published U.S. Application No. 20020006664. Published PCT applications of interest include WO 01/68836 and WO 03/010180.

### SUMMARY OF THE INVENTION

Methods and compositions are provided for selectively modulating, e.g., reducing, coding sequence expression in mammalian cells. In the subject methods, an effective amount of an RNAi agent, e.g., an interfering ribonucleic acid (such as an siRNA or shRNA) or a transcription template thereof, e.g., a DNA encoding an shRNA, is introduced into a mammalian cell. Also provided are RNAi agent pharmaceutical preparations for use in the subject methods. The subject methods and compositions find use in a variety of different applications, including academic and therapeutic applications.

### BRIEF DESCRIPTION OF THE FIGURES

FIG. 1. Test of transitive RNAi in HEK293 cells. FIG. 1A—Experimental strategy for transitive RNAi. The square indicates the original trigger siRNA, and the dashed lines indicate secondary siRNAs. Effect of siRNAs on expression of GFP fusion constructs. HEK293 cells were transfected with the indicated constructs and siRNAs and photographed by fluorescence microscopy 48 hours after transfection. FIG. 1B—Effect of siRNAs on luciferase-actin expression. Luciferase activity in cells transfected with the indicated constructs and siRNAs are shown; the values shown are the mean+standard deviation of triplicate experiments.

FIG. 2. siRNA microarray for gene silencing. (A) Experimental strategy for siRNA microarray. The desired cDNA and siRNAs are printed as individual spots on glass slides and exposed briefly to lipid before placing HEK293 cells on the printed slides in culture dish. Transfected cells are visualized using fluorescent microscopy and evaluated for the effect of RNAi. Parallel RNAi on microarrays. Fluorescence photomicrograph of cells were taken after reverse transfection of the indicated siRNA and cDNAs.

### DEFINITIONS

For convenience, certain terms employed in the specification, examples, and appended claims are collected here.

As used herein, the term “vector” refers to a nucleic acid molecule capable of transporting another nucleic acid to which it has been linked. One type of vector is a genomic integrated vector, or “integrated vector”, which can become integrated into the chromosomal DNA of the host cell. Another type of vector is an episomal vector, i.e., a nucleic acid capable of extra-chromosomal replication in an appropriate host, e.g., a eukaryotic or prokaryotic host cell. Vectors capable of directing the expression of genes to which they are operatively linked are referred to herein as “expression vectors”. In the present specification, “plasmid” and “vector” are used interchangeably unless otherwise clear from the context.

As used herein, the term “nucleic acid” refers to polynucleotides such as deoxyribonucleic acid (DNA), and, where appropriate, ribonucleic acid (RNA). The term should also be understood to include, as applicable to the embodiment being described, single-stranded (such as sense or antisense) and double-stranded polynucleotides.

As used herein, the term “gene” or “recombinant gene” refers to a nucleic acid comprising an open reading frame encoding a polypeptide of the present invention, including both exon and (optionally) intron sequences. A “recombinant gene” refers to nucleic acid encoding such regulatory polypeptides, that may optionally include intron sequences that are derived from chromosomal DNA. The term “intron” refers to a DNA sequence present in a given gene that is not translated into protein and is generally found between exons. As used herein, the term “transfection” means the introduction of a nucleic acid, e.g., an expression vector, into a recipient cell by nucleic acid-mediated gene transfer.

A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from procaryotic or eukaryotic mRNA, genomic DNA sequences from procaryotic or eukaryotic DNA, and even synthetic DNA sequences. A transcription termination sequence will usually be located 3′ to the coding sequence.

Likewise, “encodes”, unless evident from its context, will be meant to include DNA sequences that encode a polypeptide, as the term is typically used, as well as DNA sequences that are transcribed into inhibitory antisense molecules.

The term “loss-of-function”, as it refers to genes inhibited by the subject RNAi method, refers a diminishment in the level of expression of a gene (e.g., reducing expression of a gene) when compared to the level in the absence of the RNAi agent, i.e., in a cell not transfected by the RNAi agent By reducing expression is meant that the level of expression of a target gene or coding sequence is reduced or inhibited by at least about 2-fold, usually by at least about 5-fold, e.g., 10-fold, 15-fold, 20-fold, 50-fold, 100-fold or more, as compared to a control. By modulating expression of a target gene is meant altering, e.g., reducing, transcription/translation of a coding sequence, e.g., genomic DNA, mRNA etc., into a polypeptide, e.g., protein, product.

The term “expression” with respect to a gene sequence refers to transcription of the gene and, as appropriate, translation of the resulting mRNA transcript to a protein. Thus, as will be clear from the context, expression of a protein coding sequence results from transcription and translation of the coding sequence.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

As used herein, the terms “transduction” and “transfection” are art recognized and mean the introduction of a nucleic acid, e.g., an expression vector, into a recipient cell by nucleic acid-mediated gene transfer. “Transformation”, as used herein, refers to a process in which a cell's genotype is changed as a result of the cellular uptake of exogenous DNA or RNA, and, for example, the transformed cell expresses a dsRNA construct.

“Transient transfection” refers to cases where exogenous DNA does not integrate into the genome of a transfected cell, e.g., where episomal DNA is transcribed into mRNA and translated into protein.

A cell has been “stably transfected” with a nucleic acid construct when the nucleic acid construct is capable of being inherited by daughter cells.

As used herein, a “reporter gene construct” is a nucleic acid that includes a “reporter gene” operatively linked to at least one transcriptional regulatory sequence. Transcription of the reporter gene is controlled by these sequences to which they are linked. The activity of at least one or more of these control sequences can be directly or indirectly regulated by the target receptor protein. Exemplary transcriptional control sequences are promoter sequences. A reporter gene is meant to include a promoter-reporter gene construct that is heterologously expressed in a cell.

As used herein, “transformed cells” refers to cells that have spontaneously converted to a state of unrestrained growth, i.e., they have acquired the ability to grow through an indefinite number of divisions in culture. Transformed cells may be characterized by such terms as neoplastic, anaplastic and/or hyperplastic, with respect to their loss of growth control. For purposes of this invention, the terms “transformed phenotype of malignant mammalian cells” and “transformed phenotype” are intended to encompass, but not be limited to, any of the following phenotypic traits associated with cellular transformation of mammalian cells: immortalization, morphological or growth transformation, and tumorigenicity, as detected by prolonged growth in cell culture, growth in semi-solid media, or tumorigenic growth in immuno-incompetent or syngeneic animals.

As used herein, “proliferating” and “proliferation” refer to cells undergoing mitosis.

As used herein, “immortalized cells” refers to cells that have been altered via chemical, genetic, and/or recombinant means such that the cells have the ability to grow through an indefinite number of divisions in culture.

The “growth state” of a cell refers to the rate of proliferation of the cell and the state of differentiation of the cell.

“Inhibition of gene expression” refers to the absence (or observable decrease) in the level of protein and/or mRNA product from a target gene. “Specificity” refers to the ability to inhibit the target gene without manifest effects on other genes of the cell. The consequences of inhibition can be confirmed by examination of the outward properties of the cell or organism (as presented below in the examples) or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof multiple selectable markers are available that confer resistance to ampicillin, bleomycin, chloramphenicol, gentamycin, hygromycin, kanamycin, lincomycin, methotrexate, phosphinothricin, puromycin, and tetracyclin.

Depending on the assay, quantitation of the amount of gene expression allows one to determine a degree of inhibition which is greater than 10%, 33%, 50%, 90%, 95% or 99% as compared to a cell not treated according to the present invention. Lower doses of administered active agent and longer times after administration of active agent may result in inhibition in a smaller fraction of cells (e.g., at least 10%, 20%, 50%, 75%, 90%, or 95% of targeted cells). Quantitation of gene expression in a cell may show similar amounts of inhibition at the level of accumulation of target mRNA or translation of target protein. As an example, the efficiency of inhibition may be determined by assessing the amount of gene product in the cell: mRNA may be detected with a hybridization probe having a nudeotide sequence outside the region used for the inhibitory double-stranded RNA, or translated polypeptide may be detected with an antibody raised against the polypeptide sequence of that region.

### DESCRIPTION OF THE SPECIFIC EMBODIMENTS

Methods and compositions are provided for selectively modulating, e.g., reducing, coding sequence expression in mammalian cells. In the subject methods, an effective amount of an RNAi agent, e.g., an interfering ribonucleic acid (such as an siRNA or shRNA) or a transcription template thereof, e.g., a DNA encoding an shRNA, is introduced into a mammalian cell. Also provided are RNAi agent pharmaceutical preparations for use in the subject methods. The subject methods and compositions find use in a variety of different applications, including academic and therapeutic applications.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, representative methods, devices and materials are now described.

All publications mentioned herein are incorporated herein by reference for the purpose of describing and disclosing the components that are described in the publications that might be used in connection with the presently described invention.

As summarized above, the subject invention provides methods of using RNAi to selectively modulate gene expression in mammalian cells. In further describing this aspect of the subject invention, the subject methods of RNAi in mammalian cells are described first in greater detail, followed by a review of various representative applications in which the subject invention finds use as well as kits that find use in practicing the subject invention.

Methods

As indicated above, one aspect of the subject invention provides methods of employing RNAi to modulate expression of a target gene or genes in a mammalian cell, e.g., that may be present in vitro or in a mammalian host. In many embodiments, the subject invention provides methods of reducing expression of one or more target genes in a target mammalian cell or host organism including the same. By reducing expression is meant that the level of expression of a target gene or coding sequence is reduced or inhibited by at least about 2-fold, usually by at least about 5-fold, e.g., 10-fold, 15-fold, 20-fold, 50-fold, 100-fold or more, as compared to a control. In certain embodiments, the expression of the target gene is reduced to such an extent that expression of the target gene/coding sequence is effectively inhibited. By modulating expression of a target gene is meant altering, e.g., reducing, transcription/translation of a coding sequence, e.g., genomic DNA, mRNA etc., into a polypeptide, e.g., protein, product.

A feature of the subject methods is that the modulation is selective for a given target gene. By selective is meant practice of the subject methods does not give rise to “transitive RNAi,” in that sequences 5′ of the original target sequence are not silenced by practice of the subject methods.

The subject invention provides methods of modulating expression of a target gene in a mammalian cell or organism comprising the same. By “organism comprising the same” is meant a mammalian organism or host that is not an in vitro cell or cell culture, e.g., is at a stage of development that is later in time than the embryonic stage of development. As such, the host organism may be a fetus, but is generally a host organism in a post-natal stage of development, e.g., juvenile, adult, etc.

In practicing the subject methods, an effective amount of an RNAi agent is introduced into the target mammalian cell, e.g., by using conventional methods of introducing nucleic acids into a cell, such as electroporation, liposome mediated uptake, etc., by administration of the agent to the host organism to modulate expression of a target gene in a desirable manner, e.g., to achieve the desired reduction in target cell gene expression.

By RNAi agent is meant an agent that modulates expression of a target gene by a RNA interference mechanism. The RNAi agents employed in one embodiment of the subject invention are small ribonucleic acid molecules (also referred to herein as interfering ribonucleic acids), i.e., oligoribonucleotides, that are present in duplex structures, e.g., two distinct oligoribonucleotides hybridized to each other or a single ribooligonucleotide that assumes a small hairpin formation to produce a duplex structure. By oligoribonucleotide is meant a ribonucleic acid that does not exceed about 100 nt in length, and typically does not exceed about 75 nt length, where the length in certain embodiments is less than about 70 nt. Where the RNA agent is a duplex structure of two distinct ribonucleic acids hybridized to each other, e.g., an siRNA (such as d-siRNA as described in copending application Ser. No. 60/377,704; the disclosure of which is herein incorporated by reference), the length of the duplex structure typically ranges from about 15 to 30 bp, usually from about 15 to 29 bp, where lengths between about 20 and 29 bps, e.g., 21 bp, 22 bp, are of particular interest in certain embodiments. Where the RNA agent is a duplex structure of a single ribonucleic acid that is present in a hairpin formation, i.e., a shRNA, the length of the hybridized portion of the hairpin is typically the same as that provided above for the siRNA type of agent or longer by 4-8 nucleotides. The weight of the RNAi agents of this embodiment typically ranges from about 5,000 daltons to about 35,000 daltons, and in many embodiments is at least about 10,000 daltons and less than about 27,500 daltons, often less than about 25,000 daltons.

In certain embodiments, instead of the RNAi agent being an interfering ribonucleic acid, e.g., an siRNA or shRNA as described above, the RNAi agent may encode an interfering ribonucleic acid, e.g., an shRNA, as described above. In other words, the RNAi agent may be a transcriptional template of the interfering ribonucleic acid. In these embodiments, the transcriptional template is typically a DNA that encodes the interfering ribonucleic acid. The DNA may be present in a vector, where a variety of different vectors are known in the art, e.g., a plasmid vector, a viral vector, etc.

As indicated above, the RNAi agent can be introduced into the target mammalian cell(s) using any convenient protocol, where the protocol will vary depending on whether the target cells are in vitro or in vivo.

Where the mammalian target cells are in vivo, the RNAi agent can be administered to the mammalian host using any convenient protocol, where the protocol employed is typically a nucleic acid administration protocol, where a number of different such protocols are known in the art. The following discussion provides a review of representative nucleic acid administration protocols that may be employed. The nucleic acids may be introduced into tissues or host cells by any number of routes, including viral infection, microinjection, or fusion of vesicles. Jet injection may also be used for intramuscular administration, as described by Furth et al. (1992), Anal Biochem 205:365-368. The nucleic acids may be coated onto gold microparticles, and delivered intradermally by a particle bombardment device, or “gene gun” as described in the literature (see, for example, Tang et al. (1992), Nature 356:152-154), where gold microprojectiles are coated with the DNA, then bombarded into skin cells. Expression vectors may be used to introduce the nucleic acids into a cell. Such vectors generally have convenient restriction sites located near the promoter sequence to provide for the insertion of nucleic acid sequences. Transcription cassettes may be prepared comprising a transcription initiation region, the target gene or fragment thereof, and a transcriptional termination region. The transcription cassettes may be introduced into a variety of vectors, e.g. plasmid; retrovirus, e.g. lentivirus; adenovirus; and the like, where the vectors are able to transiently or stably be maintained in the cells, usually for a period of at least about one day, more usually for a period of at least about several days to several weeks.

For example, the RNAi agent can be fed directly to, injected into, the host organism containing the target gene. The agent may be directly introduced into the cell (i.e., intracellularly); or introduced extracellularly into a cavity, interstitial space, into the circulation of an organism, introduced orally, etc. Methods for oral introduction include direct mixing of RNA with food of the organism. Physical methods of introducing nucleic acids include injection directly into the cell or extracellular injection into the organism of an RNA solution. The agent may be introduced in an amount which allows delivery of at least one copy per cell. Higher doses (e.g., at least 5, 10, 100, 500 or 1000 copies per cell) of the agent may yield more effective inhibition; lower doses may also be useful for specific applications.

In certain embodiments, a hydrodynamic nucleic acid administration protocol is employed. Where the agent is a ribonucleic acid, the hydrodynamic ribonucleic acid administration protocol described in detail below is of particular interest. Where the agent is a deoxyribonucleic acid, the hydrodynamic deoxyribonucleic acid administration protocols described in Chang et al., J. Virol. (2001) 75:3469-3473; Liu et al., Gene Ther. (1999) 6:1258-1266; Wolff et al., Science (1990) 247: 1465-1468; Zhang et al., Hum. Gene Ther. (1999) 10:1735-1737: and Zhang et al., Gene Ther. (1999) 7:1344-1349; are of interest.

Additional nucleic acid delivery protocols of interest include, but are not limited to: those described in U.S. Patents of interest include U.S. Pat. Nos. 5,985,847 and 5,922,687 (the disclosures of which are herein incorporated by reference); WO/11092;. Acsadi et al., New Biol. (1991) 3:71-81; Hickman et al., Hum. Gen. Ther. (1994) 5:1477-1483; and Wolff et al., Science (1990) 247: 1465-1468; etc.

Depending n the nature of the RNAi agent, the active agent(s) may be administered to the host using any convenient means capable of resulting in the desired modulation of target gene expression. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. As such, administration of the agents can be achieved in various ways, including oral, buccal, rectal, parenteral, intraperitoneal, intradermal, transdermal, intracheal, etc., administration.

In pharmaceutical dosage forms, the agents may be administered alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Those of skill in the art will readily appreciate that dose levels can vary as a function of the specific compound, the nature of the delivery vehicle, and the like. Preferred dosages for a given compound are readily determinable by those of skill in the art by a variety of means.

Introduction of an effective amount of an RNAi agent into a mammalian cell as described above results in a modulation of target gene(s) expression, e.g., a reduction of target gene(s) expression, as described above.

The above described methods work in any mammalian cell, where representative mammal cells of interest include, but are not limited to cells of: ungulates or hooved animals, e.g., cattle, goats, pigs, sheep, etc.; rodents, e.g., hamsters, mice, rats, etc.; lagomorphs, e.g., rabbits; primates, e.g., monkeys, baboons, humans, etc.; and the like.

The above-described methods find use in a variety of different applications, representative types of which are now described in greater detail below.

Utility

The subject methods find use in a variety of different applications, where representative applications include both academic/research applications and therapeutic applications. Each of these types of representative applications is described more fully below.

Academic/Research Applications

The subject methods find use in a variety of different types of academic, research applications, in which one desires to selectively modulate expression of one or more target genes (coding sequences) in a mammalian cell or host that includes the same, e.g., to determine the function of a target gene/coding sequence in a mammalian host. The subject methods find particular use in “loss-of-function” type assays, where one employs the subject methods to reduce or decrease or inhibit expression of one or more target genes/coding sequences in a mammalian cell.

As such, one representative utility of the present invention is as a method of identifying gene function in a mammalian cell, where an RNAi agent is introduced into a mammal cell according to the present invention in order to inhibit the activity of a target gene of previously unknown function. Instead of the time consuming and laborious isolation of mutants by traditional genetic screening, functional genomics using the subject methods determines the function of uncharacterized genes by administering an RNAi agent to reduce the amount and/or alter the timing of target gene activity. Such methods can be used in determining potential targets for pharmaceutics, understanding normal and pathological events associated with development, determining signaling pathways responsible for postnatal development/aging, and the like. The increasing speed of acquiring nucleotide sequence information from genomic and expressed gene sources, including total sequences for mammalian genomes, can be coupled with use of the subject methods to determine gene function in a live mammalian organism. The preference of different organisms to use particular codons, searching sequence databases for related gene products, correlating the linkage map of genetic traits with the physical map from which the nucleotide sequences are derived, and artificial intelligence methods may be used to define putative open reading frames from the nucleotide sequences acquired in such sequencing projects.

A simple representative assay inhibits gene expression according to the partial sequence available from an expressed sequence tag (EST). Functional alterations in growth, development, metabolism, disease resistance, or other biological processes would be indicative of the normal role of the ESTs gene product. The function of the target gene can be assayed from the effects it has on the mammal when gene activity is inhibited.

If a characteristic of an organism is determined to be genetically linked to a polymorphism through RFLP or QTL analysis, the present invention can be used to gain insight regarding whether that genetic polymorphism might be directly responsible for the characteristic. For example, a fragment defining the genetic polymorphism or sequences in the vicinity of such a genetic polymorphism can-be employed to produce an RNAi agent, which agent can then be administered to the mammal, and whether an alteration in the characteristic is correlated with inhibition can be determined.

The present invention is useful in allowing the inhibition of essential genes. Such genes may be required for organism viability at only particular stages of development or cellular compartments. The functional equivalent of conditional mutations may be produced by inhibiting activity of the target gene when or where it is not required for viability. The invention allows addition of an RNAi agent at specific times of development and locations in the organism without introducing permanent mutations into the target genome.

In situations where alternative splicing produces a family of transcripts that are distinguished by usage of characteristic exons, the present invention can target inhibition through the appropriate exons to specifically inhibit or to distinguish among the functions of family members. For example, a hormone that contained an alternatively spliced transmembrane domain may be expressed in both membrane bound and secreted forms. Instead of isolating a nonsense mutation that terminates translation before the transmembrane domain, the functional consequences of having only secreted hormone can be determined according to the invention by targeting the exon containing the transmembrane domain and thereby inhibiting expression of membrane-bound hormone.

Therapeutic Applications

The subject methods also find use in a variety of therapeutic applications in which it is desired to selectively modulate, e.g., one or more target genes in a whole mammal or portion thereof, e.g., tissue, organ, etc, as well as in mammalian cells present therein. In such methods, an effective amount of an RNAi active agent is administered to the host mammal or mammalian cell. By effective amount is meant a dosage sufficient to selectively modulate expression of the target gene(s), as desired. As indicated above, in many embodiments of this type of application, the subject methods are employed to reduce/inhibit expression of one or more target genes in the host in order to achieve a desired therapeutic outcome.

Depending on the nature of the condition being treated, the target gene may be a gene derived from the cell, an endogenous gene, a pathologically mutated gene, e.g. a cancer causing gene, a transgene, or a gene of a pathogen which is present in the cell after infection thereof. Depending on the particular target gene and the dose of RNAi agent delivered, the procedure may provide partial or complete loss of function for the target gene. Lower doses of injected material and longer times after administration of RNAi agent may result in inhibition in a smaller fraction of cells.

The subject methods find use in the treatment of a variety of different conditions in which the modulation of target gene expression in a mammalian host is desired. By treatment is meant that at least an amelioration of the symptoms associated with the condition afflicting the host is achieved, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g. prevented from happening, or stopped, e.g. terminated, such that the host no longer suffers from the condition, or at least the symptoms that characterize the condition.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

The present invention is not limited to modulation of expression of any specific type of target gene or nudeotide sequence. Representative classes of target genes of interest include but are not limited to: developmental genes (e.g., adhesion molecules, cyclin kinase inhibitors, cytokinesaymphokines and their receptors, growth/differentiation factors and their receptors, neurotransmitters and their receptors); oncogenes (e.g., ABLI, BCLI, BCL2, BCL6, CBFA2, CBL, CSFIR, ERBA, ERBB, EBRB2, ETSI, ETS1, ETV6, FOR, FOS, FYN, HCR, HRAS, JUN, KRAS, LCK, LYN, MDM2, MLL, MYB, MYC, MYCLI, MYCN, NRAS, PIM 1, PML, RET, SRC, TALI, TCL3, and YES); tumor suppressor genes (e.g., APC, BRCA 1, BRCA2, MADH4, MCC, NF 1, NF2, RB 1, TP53, and WTI); and enzymes (e.g., ACC synthases and oxidases, ACP desaturases and hydroxylases, ADP-glucose pyrophorylases, ATPases, alcohol dehydrogenases, amylases, amyloglucosidases, catalases, cellulases, chalcone synthases, chitinases, cyclooxygenases, decarboxylases, dextrinases, DNA and RNA polymerases, galactosidases, glucanases, glucose oxidases, granule-bound starch synthases, GTPases, helicases, hemicellulases, integrases, inulinases, invertases, isomerases, kinases, lactases, Upases, lipoxygenases, lyso/ymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, and xylanases); chemokines (e.g. CXCR4, CCR5), the RNA component of telomerase, vascular endothelial growth factor (VEGF), VEGF receptor, tumor necrosis factors nuclear factor kappa B, transcription factors, cell adhesion molecules, Insulin-like growth factor, transforming growth factor beta family members, cell surface receptors, RNA binding proteins (e.g. small nudeolar RNAs, RNA transport factors), translation factors, telomerase reverse transcriptase); etc.

Kits

Also provided are reagents and kits thereof for practicing one or more of the above-described methods. The subject reagents and kits thereof may vary greatly. Typically, the kits at least include an RNAi agent as described above.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The following examples are offered by way of illustration and not by way of limitation.

### EXPERIMENTAL

I. Materials and Methods

A. Cells and Reagents

Human embryonic kidney (HEK) 293 cells (American Tissue Culture Collection) and 293-derived Phoenix amphotropic packaging cell line (G. Nolan, Stanford) are obtained from the indicated sources. Single stranded dTdT RNA oligonucleotides (Dharmacon) were annealed to generate siRNA. Stable GFP-expressing Phoenix cells were produced by transient transfection of pMIGR (gift of W. Pear, U. Pennsylvania) into amphotropic Phoenix cells and followed by two rounds of fluorescence-activated cell sorting (FACS) selection of GFP+ cells. The resultant cells were>95% GFP+ and remained so subsequently without additional selection. Constructs: eGFP-N3, dsRED, YFP-actin, and pSEAP2-control (Clontech), and pGL3 luciferase (Promega) were obtained from indicated sources. The Xhol-BamHI actin fragment from YFP-actin was released by restriction digestion and cloned into eGFP-N3 and pGL3-control to generate ActinS-GFP, ActinAS-GFP and Luciferase-actin constructs.

B. siRNA Experiments

Expression constructs and siRNAs were transfected using Lipofectamine 2000 (Invitrogen) as described in Elbashir et al., Nature (2001) 411:494-498. GFP expression was assayed by either FACS or fluorescence microscopy 48-72 hours after transfection. Transfection efficiency was normalized by dividing GFP or luciferase fluorecent units with the secreted placental alkaline phosphatase activity generated from cotransfected pSEAP2-control plasmid.

C. Microarray Procedures

Messenger RNA was purified using Fastrack (Invitrogen) following manufacturer's instructions. A reference mRNA standard prepared by pooling RNA from eleven cell lines were used in all experiments. Microarray techniques were as described Perou et al., Nature (2000) 406:747-752. For the siRNA arrays, the annealed RNA duplexes were precipitated in ethanol and resuspended in water for array printing. Complementary DNA and siRNA were dissolved in 0.2% gelatin and printed on amine-covered glass slides (Corning) using a robotic arrayer, and reverse trransfection of HEK293 cells was performed using Effectene (Qiagen) as described Ziauddin & Sabatini, Nature (2001) 411:107-110. Reverse transfected cells were visualized by digital phase contrast and fluorescence microscopy (Canon).

D. Statistical Methods

The gene expression data from 3 sets of siRNA experiments were derived from 27 microarrays and were analyzed separately in 3 data sets. In each data set, genes were considered well-measured if the reference channel had >1.5 fold of signal intensity over background and was present for >80% of data set. The three sets of genes were each analyzed by multi-class comparison in SAM Tusher et al., Proc. Nat'l Acad. Sci. USA (2001) 98:5116-5121, and the false discovery rate of the top 10 SAM-selected genes was calculated. The top 10 genes from each data set were collated, and the expression data of this set of 30 genes from each data set was retrieved and grouped by hierarchical clustering Eisen et al., Proc. Nat'l Acad. Sci. USA (1998) 95:14863-14868.

E. Silencing of a Model Gene by siRNAs

Silencing of transiently expressed and integrated GFP gene by siRNAs. Sequences of the siRNAs used were:

| | | | |
|---|---|---|---|
| 5′ | CUACAACAGCCACAACGUCdTdT | 3′ | (SEQ ID NO:01) |
| | dTdTGAUGUUGUCGGUGUUGCAG | | |
| 5′ | CAACAUCUCGACACCAGCAdTdT | 3′ | (SEQ ID NO:02) |
| | dTdTGUUGUAGAGCUGUGGUCGU | | |
| 5′ | CAGCCACAACGUCUAUAUCdTdT | 3′ | (SEQ ID NO:03) |
| | dTdTGUCGGUGUUGCAGAUAUAG | | |
| 5′ | ACAGACCACCGUGUCUAACdTdT | 3′ | (SEQ ID NO:04) |
| | dTdTUGUCUGGUGGCACAGAUUG | | |

For silencing of transiently transfected GFP, 0.3 μg of pGFP was transfected with 1 μg of pSEAP2-control-and 12 picomoles of the indicated siRNA in HEK293 cells. For silencing of an integrated GFP gene, HEK293- derived Phoenix cells expressing GFP after retroviral transduction (Methods) were transfected with the 12 picomoles of the indicated siRNA and 1 μg of pSEAP2-control. GFP expression was determined by FACS 48 hours (transient GFP target) or 72 hours (integrated GFP target) after transfection. The mean fluorescence intensity was normalized for transfection efficiency by the alkaline phosphatase activity of pSEAP2-control (Methods). The experiments were done in triplicate, and the means (+standard deviation) of GFP fluorescence intensity relative to mock transfected cells (no siRNA) are shown. Fluorescence photomicroscopy and FACS plots of cells stably expressing GFP and transfected with the indicated siRNAs were also obtained.

F. Global Gene Expression Changes Associated with RNAi.

Global gene expression patterns in 3 siRNA experiments were analyzed; in each set the gene expression of cells which were mock transfected (no siRNA), transfected with GFP siRNA, or cognate control siRNA were determined in parallel in triplicate. Data sets: (El) HEK293 cells with transiently expressed GFP target treated with E1, C1, or no siRNA; (E2) HEK293 cells with transiently expressed GFP target treated with E2, C2, or no siRNA; (stable) Phoenix cells stably expressing an integrated GFP gene treated with El, Cl, or no siRNA. Genes that had signal intensity >1.5 fold of the local spot element background in the reference channel and were present for >80% of the data set were considered well measured. A summary of the results is provided below:

| Data | Number of Well- | Number of Genes | FDR for top |
|---|---|---|---|
| Set | Measured Genes | with FDR < 0.05 | 10 genes |
| E1 | 17,891 | 0 | 0.19 |
| E2 | 24,048 | 0 | 0.30 |
| Stable | 19,655 | 0 | 0.22 |

The number of well-measured genes are shown on the second column; these genes were analyzed in the multi-class comparison using SAM. The number of genes which had an estimated false discovery rate (FDR) of <0.05 and the FDR of the top 10 performing genes for each data set are shown on the right two columns, Minimal gene expression changes associated with siRNA-mediated RNAi were observed. The 10 genes with the most consistent changes in expression in response to the experimental manipulation, in each of the 3 siRNA experiments, were collated into a non-redundant gene list. The expression changes of this group of genes in all experiments were displayed in matrix format. The expression ratios were mean-centered within each data set.

II. Results

A. Global View of Gene Silencing by siRNA

To evaluate the specificity of siRNA, we used a target gene that has no normal role or known physiological effects in the cell, so that its presence or absence would not otherwise perturb the transcriptome. We chose the enhanced green fluoresecent protein (GFP) of Aequoria Victoria as a model target because the protein level is easily monitored, it is an exogenous protein that has no normal function in human cells, and it is relatively nontoxic and known to be well tolerated in normal development. As previously reported by Elbashir et al., Nature (2001) 411: 3494-498, transient transfection of HEK293 cells with GFP and the two siRNAs directed toward GFP sequences (termed E1 and E2) suppressed the level of GFP activity by over 80%, but cotransfection of GFP with scrambled siRNAs matched for nucleotide content (termed C1 and C2, respectively) did not affect GFP activity compared to mock transfected cells, which were not exposed to siRNA. C1 and C2 did not have significant homology to any human gene or expressed sequence tags (EST) in the NR and EST database when analyzed with Blast program in NCBI. The transfection efficiency was above 80% as judged by GFP fluorescence. To address the specificity of RNAi against an integrated and nuclear gene, we established a population of cells stably expressing a GFP gene that was introduced by retroviral transduction (Methods). Transfection of these stable GFP-expressing cells with the E1 siRNA silenced GFP expression by more than 70%, but GFP expression was unaffected by mock or C1 transfection.

The global gene expression patterns of cells after mock transfection, silencing of transiently expressed or stably expressed GFP by E1 or E2 siRNA, and control silencing by C1 or C2 siRNA were determined using human cDNA microarrays. The microarrays contained approximately 43,000 elements, corresponding to approximately 36,000 genes based on Unigene-data. Because even small differences in cell passage or media metabolism can lead to differences in global gene expression pattern, control and siRNA experiments were always performed in parallel in sets of three and in triplicate as described above. To search for gene expression responses associated with RNA interference, we performed a statistical test (SAM) to identify genes whose expression varied accordingly in response to the experimental manipulations we tested, Tusher et al., supra. SAM is a permutation-based technique that permits the estimation of a false discovery rate (FDR) for set of genes identified Tusher et al., supra. The FDR is analogous to p-value in standard statistical tests, but the FDR can accommodate the effects of non-normal distribution in the data and multiple testing Tusher et al., supra. For each of the three sets of gene expression data, none of approximately 20,000 well-measured mRNAs was consistently affected by the siRNA treatments, with a FDR <0.05 . The 10 genes that showed the most consistent changes in expression with the experimental manipulations had estimated FDRs that ranged from 0.19 to 0.30 in the three experiments. The top 10 genes identified by SAM in all three data sets were noted. We note that the genes that showed the largest apparent responses in the three sets of experiments did not overlap, and the magnitude of the changes in expression of any of these genes was small (mostly less than 2 fold). Moreover, these small variations in gene expression did not consistently distinguish the siRNA-silenced samples from the mock treated samples. Among all of the genes that showed variation in expression in the experiments identifying either transiently or stably GFP, none showed a consistent response pattern. Thus, we believe that the small observed variations are likely to be due to experimental noise, rather than resulting from the siRNA treatment.

Collectively, we interpret these results to indicate that no consistent “off-target” gene expression perturbation is associated with the process of siRNA-mediated gene silencing. To the detectable limits of our cDNA array method, siRNA-mediated gene silencing in the tested cells appears to be highly sequence-specific.

B. Evaluation of Transitive RNAi in Human Cells

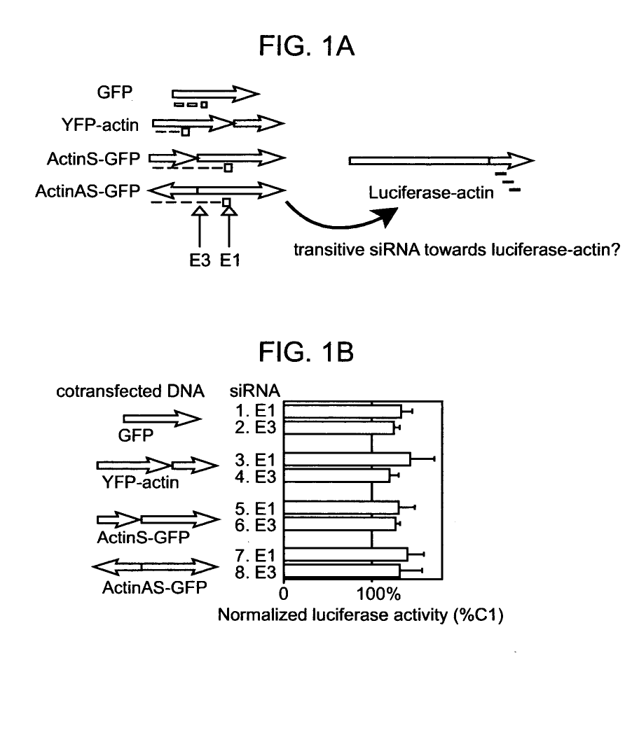

Although siRNAs appear to be highly sequence-specific, the extension of RNAi-mediated silencing to sequences 5′ to the mRNA sequence complementary to the siRNA could generate secondary siRNAs that could potentially target other mRNAs with sequence similarity. Such a phenomenon, termed “transitive RNAi” has been shown to occur in C. elegans (Sijen et al., Cell (2001) 107:465-476. To test for the occurrence of transitive RNAi in human cells, we cotransfected into HEK 293 cells two sets of reporter genes (GFP/YFP and luciferase) with sequence overlap engineered by fusing a sequence for the actin gene to both sets of constructs (FIG. 1A). We used siRNA E1 to target the first reporter genes (GFP or YFP, which contain the same cognate sequence) and verified the RNA silencing by monitoring the fluorescence of transfected HEK293 cells. If transitive RNAi were active in 293 cells, silencing of GFP/YFP-actin fusion mRNA should generate secondary siRNAs targeting the actin sequences and thereby initiate the silencing of the second reporter gene, luciferase-actin, resulting in diminished luciferase activity. We tested the transitive effects of silencing GFP expressed alone or in the form of fusion transcripts with actin fused at either the 3′ end of yellow fluorescent protein (YFP-actin), or at the 5′ end of GFP in both orientations (ActinS-GFP, ActinAS-GFP). Fluorescent microscopy confirmed that siRNA-mediated RNA silencing of the primary target gene was achieved for all four pairs of different fluorescent proteins. In all four experiments, the luciferase activity in the cells silenced by GFP siRNA (E1) was not lower than that in cells treated with control siRNA (C1) (FIG. 1B). These results show that transitive RNAi, at least on the scale demonstratable in Drosophila extract and C. elegans, does not occur during siRNA-mediated silencing in 293 cells. This result may be related to the relatively inefficient silencing mediated by siRNA in mammalian cells compared to that seen in Drosophila or C. elegans.

C. siRNA-Mediated RNAi on Microarrays

The rapidly expanding catalogue of eukaryotic genes, from a diverse and expanding array of sequencing projects, presents scientists with the challenge of understanding the biological roles of each newly identified gene. Recent advances in RNAi technology in lower organisms have already yielded powerful insights into the functions of many genes and their protein products. RNAi has been successfully applied to systemic analysis of the C. elegans genome, but the effort still depends on the analysis of the phenotypes of individual worms resulting from disruption of one gene at one time. The recent development of high-throughput cDNA transfection on microarrays (Ziauddin & Sabatini et al., supra) provides a model for the use of siRNAs on high-density microarrays to perform RNAi in mammalian cells in a highly parallel fashion.

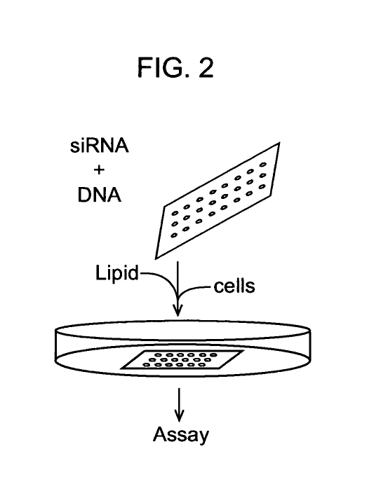

We tested the feasibility of siRNAs-mediated RNAi on microarrays (FIG. 2). DNAs encoding GFP, dsRED, and siRNAs were spotted in the desired combinations on amine glass slides using a robotic arrayer. We hypothesized that in the presence of lipids, siRNA would complex with the DNA printed on the slide and form liposomes containing both reagents. Expression of dsRED served as an internal control for reverse transfection and localization of the printed spots. After air drying, the printed arrays were exposed to Effectene briefly and placed in a tissue culture dish. HEK293 cells were then plated on the arrays and cultured in Petri dish. The cells were examined with fluorescence microscopy 72 hour later. As shown in FIG. 4B, HEK293 cells expressed dsRED in all the cell clusters above the printed spots after reverse transfection. In contrast, GFP expression was readily apparent in the control spots and selectively decreased in the presence of the siRNA E1, complimentary to GFP mRNA, but not in the presence of the control siRNA C1 (data not shown). The merged image allowed quick detection of specific RNAi effect by the red shift of the affected cell clusters.

These results demonstrate that siRNA-mediated gene silencing can be adapted to microarray format. By arraying different siRNAs on microarrays, one can generate a large panels of cells silenced for different genes for highly parallel tests of gene function.

III. Discussion

Using DNA microarrays to profile global gene expression, we have demonstrated that siRNA-mediated gene silencing has exquisite sequence specificity for the target mRNA and does not induce detectable secondary changes in the global gene expression pattern. We tested for transitive RNAi using paired, highly-expressed transcripts with overlapping sequence identity, conditions which easily afforded detection of transitive RNAi in C. elegans (Sijen et al., supra). The lack of robust transitive RNAi in human cells is consistent with published reports of selective targeting of splicing isoforms using siRNA, the lack of an obvious RNA-dependent RNA polymerase in the human genome, and the dispensability of priming activity of siRNAs for RNAi in mammalian cells. These results provide further confirmation for using siRNA-mediated RNAi as a research and therapeutic tool. The high specificity observed in these experiments, increases the confidence with which phenotypes observed with siRNA-mediated silencing can be ascribed to the targeted genes. The results confirm the position that siRNA-based therapeutic agents have useful molecular specificity. Because the process of siRNA-mediated silencing does not appear, in general, to produce nonspecific gene expression changes, global changes of gene expression patterns provide an assay with which to study and annotate the function of unknown genes, especially based on comparisons to gene expression patterns of mutants in known pathways (Hughes et al., Cell (2002) 102: 109-126).

Application of siRNA technology on a genome-wide scale could be significantly accelerated by a platform for delivering siRNAs and screening the resulting phenotypes in a high throughput fashion. We have examined the feasibility of arraying siRNAs on glass microarrays and performing RNAi experiments by reverse transfection. This method provides a practical means to conduct highly parallel RNAi experiments in mammalian cells in a spatially-addressable fashion. Approximately 10,000 array elements can be accommodated on a standard glass microscope slide in the format that we tested. As we have demonstrated using two reporter genes to monitor transfection and gene silencing separately, arrays of cells silenced for different genes may be screened for altered morphology, activation of signal transduction pathways using specific reporter genes, or expression of endogenous markers using immunofluorescence. The microarray format also lends itself to comprehensively testing the effect of silencing various combinations of genes within a family and thereby confronting the issues of redundancy and compensation that frequently arise in mammalian genetics.

It is evident from the above results and discussion that the subject invention provides a viable way of using RNAi agents in mammalian cells organisms, where the subject methods and compositions can be employed for a variety of different academic and therapeutic applications. A feature of the subject methods is that they provide for selective RNAi modulation of gene expression, which does not suffer from the phenomenon of “transitive RNAi.” As such, the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method of selectively reducing expression of a coding sequence in a mammalian target cell, said method comprising:
introducing into said mammalian target cell an effective amount of an RNAi agent specific for said coding sequence to selectively reduce expression of said coding sequence.

2. The method according to claim 1, wherein said RNAi agent is an interfering ribonucleic acid.

3. The method according to claim 2, wherein said interfering ribonucleic acid is a siRNA.

4. The method according to claim 2, wherein said interfering ribonucleic acid is a shRNA.

5. The method according to claim 1, wherein said RNAi agent is a transcription template of an interfering ribonucleic acid.

6. The method according to claim 5, wherein said transcription template is a deoxyribonucleic acid.

7. The method according to claim 6, wherein said deoxyribonucleic acid encodes a shRNA.

8. The method according to claim 1, wherein said mammalian cell is present in vitro.

9. The method according to claim 1, wherein said mammalian cell is present in vivo.

10. A pharmaceutical preparation comprising an RNAi agent in a pharmaceutically acceptable delivery vehicle.

11. The pharmaceutical preparation according to claim 10, wherein said preparation further comprises an RNAse inhibitor.

12. A kit for use in practicing the method of claim 1, said kit comprising:
(a) a pharmaceutical preparation comprising an RNAi agent in a pharmaceutically acceptable delivery vehicle; and
(b) instructions for practicing the method of claim 1.

13. The kit according to claim 14, wherein said kit further comprises an RNAse inhibitor.
